(19) United States
(12) Patent Application Publication
(10) Pub. No.: US 2006/0099626 A1
(43) Pub. Date: May 11, 2006

(54) DNA-templated combinatorial library device and method for use

(76) Inventors: Pehr B. Harbury, San Francisco, CA (US); Stephen Jarrett Wrenn, San Francisco, CA (US); Rebecca Maile Weisinger, Belmont, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/262,300

(22) Filed: Oct. 27, 2005

Related U.S. Application Data
(60) Provisional application No. 60/622,752, filed on Oct. 27, 2004.

(51) Int. Cl. C12Q 1/68; C12M 1/34; C40B 40/08

(52) U.S. Cl. 435/6; 435/287.2

(57) ABSTRACT

The present invention provides a device and method for synthesizing nucleic acid-templated combinatorial chemical libraries. The device includes a splitting filter having an array of immobilized capture nucleic acids and a chemical coupling filter having an array of non-specific binding features, wherein the plates are positioned to provide for alignment between the capture sites of the first filter and the non-specific binding features of the second filter plate. The molecules bound to the splitting filter can be transferred to the chemical coupling filter and then reacted with site-specific reagents to chemically modify the bound molecules.

### CROSS-REFERENCE

This application claims the benefit of U.S. Provisional Application No. 60/622,752, filed Oct. 27, 2004, which application is incorporated herein by reference.

### FIELD OF THE INVENTION

The present invention relates to a device and method for synthesizing a DNA-templated combinatorial chemistry library of compounds using the device.

### BACKGROUND OF THE INVENTION

Drug discovery generally proceeds by fractionation of natural extracts with medicinal properties, or by serial screening of random chemical collections for molecules with a biological activity. The resulting lead compounds are subjected to extensive chemical modification in order to generate variants that work well in animals. This process is slow, expensive, and requires an enormous infrastructure.

Efforts to accelerate this process by combinatorial-library strategies have led to a radically different strategy for molecular discovery. One such approach, collectively termed in vitro evolution, is based on diverse-libraries of gene products, each of which is physically associated with a corresponding DNA blueprint. The entire library is tested in parallel using a functional selection, such as binding to an immobilized macromolecule, to physically isolate gene products with a desired functional property.

Although application of in vitro evolution approaches to drug discovery would likely prove to be very effective, it has not been possible. Classical in vitro evolution techniques have been restricted to biopolymer libraries encoded by DNA genes. The product nucleic acid and peptide ligands lack the membrane permeability and metabolic stability required of medicines.

Co-owned patent application WO 00/23458, for “DNA Templated Combinatorial Library Chemistry,” published Apr. 27, 2000, based on PCT/US99/24494, and incorporated herein by reference, discloses a method and composition for iterative synthesis and screening of small-molecule compound libraries in which a nucleic acid tag comprising catenated coding positions directs the synthesis of the compound to which the nucleic acid tag is covalently attached. Since the tag is a DNA molecule, the tag can be amplified biochemically and used to direct the synthesis of a large quantity of the corresponding small molecule. The method allows for synthesis of a large number of different small-molecule compounds in a combinatorial library by way of a split and combine synthesis strategy, where synthesis is directed by the nucleic acid tag. According to an important advantage of the method, selected molecules of interest can be enriched by (i) amplifying the associated nucleic acid tags, e.g., by PCR, and (ii) using the amplified tags to direct the synthesis of molecules of interest corresponding to those tags. The method also allows the “space” around library compounds of interest to be explored for more active compounds, by gene-shuffling methods applied to the nucleic acid tags.

Ideally, in building large robust drug-discovery libraries, it is useful to build small compounds using 3-6 step synthetic methods. Therefore, in order to achieve high library diversity each reaction step must encompass a large number of different compound reactions, e.g., 100-2,000 or more different reactions at each reactions step, thus achieving, for example, 4×10⁸ (four reaction steps, 100 different reactions/step) or 4×10¹² (four reaction steps, 1,000 different reactions/steps) for the total library size.

Accordingly, there is a need in the art to provide a device and method that would expedite the synthesis and screening of large DNA-templated combinatorial libraries where large numbers of reactions, e.g., 50-2,000 or more, are carried out at each reaction step, e.g., for building small-molecule libraries that involve a small number of successive synthesis steps, typically 3-6 steps. The present invention addresses these needs.

Relevant Literature

Halpin et al., PLoS Biol, 2004. 2(7): p. E173; Halpin et al., PLoS Biol, 2004. 2(7): p. E174; Halpin et al., PLoS Biol, 2004. 2(7): p. E175; Gartner et al., Science, 2004. 305(5690): p. 1601-5; Kerns et al., J Pharm Sci, 2001. 90(11): p. 1838-58; Di et al., J Pharm Sci, 2004. 93(6): p. 1537-44; Druker et al., N Engl J Med, 2001. 344(14): p. 1031-7; Druker et al., N Engl J Med, 2001. 344(14): p. 1038-42; Gorre et al., Science, 2001. 293(5531): p. 876-80; Shah et al., Science, 2004. 305(5682): p. 399-401; Shah et al., Cancer Cell, 2002. 2(2): p. 117-25; Deng et al., Blood, 2001. 97(11): p. 3491-7; Lipinski, et al., ADVANCED DRUG DELIVERY REVIEWS, 1997. 23(1-3): p. 3-25; Figliozzi et al., Methods Enzymol, 1996. 267: p. 437-47; Miller et al., Drug Development Research, 1995. 35: p. 20-32; Wang et al., Biopharm Drug Dispos, 1999. 20(2): p. 69-75; Zuckermann et al., J Med Chem, 1994. 37(17): p. 2678-85; Goodson et al., Antimicrob Agents Chemother, 1999. 43(6): p. 1429-34; Garcia-Martinez et al., Proc Natl Acad Sci U S A, 2002. 99(4): p. 2374-9; Wang et al., J Biol Chem, 1985. 260(1): p. 64-71; Foulkes et al., J Biol Chem, 1985. 260(13): p. 8070-7; Rees-Jones et al., J Virol, 1988. 62(3): p. 978-86; Pritchard et al., Biochem J, 1989. 257(2): p. 321-9; Lydon et al., Oncogene Res, 1990. 5(3): p. 161-73; Rayter et al., Methods Enzymol, 1991. 200: p. 596-604; Garcia et al., J Biol Chem, 1993. 268(33): p. 25146-51; Kapust et al., Protein Sci, 1999. 8(8): p. 1668-74; Minoguchi et al., Mol Immunol, 1994. 31(7): p. 519-29; Edelhoch et al., Biochemistry, 1967. 6(7): p. 1948-54; Gordon et al., J Med Chem, 1994. 37(10): p. 1385-401; Daley et al., Proc Natl Acad Sci USA, 1988. 85(23): p. 9312-6; Casnellie et al., Methods Enzymol, 1991. 200: p. 115-20; Knockaert et al., Chem Biol, 2000. 7(6): p. 411-22; Godl et al., Proc Natl Acad Sci USA, 2003. 100(26): p. 15434-9; Lolli et al., Proteomics, 2003. 3(7): p. 1287-98; Backes et al., Curr Opin Chem Biol, 1997. 1(1): p. 86-93; Fukuyama et al., Tetrahedron Letters, 1995. 36(36): p. 6373-6374; Huron et al., Clin Cancer Res, 2003. 9(4): p. 1267-73; Pidgeon et al., J Med Chem, 1995. 38(4): p. 590-4; Valko et al., J Pharm Sci, 2000. 89(8): p. 1085-96; and Houston et al., Biochem Pharmacol, 1994. 47(9): p. 1469-79.

### SUMMARY OF THE INVENTION

The present invention provides a device and method for synthesizing nucleic acid-templated combinatorial chemical libraries. The device includes a splitting filter having an array of immobilized capture nucleic acids and a chemical coupling filter having an array of non-specific binding features, wherein the plates are positioned to provide for alignment between the capture sites of the first filter and the non-specific binding features of the second filter plate. The molecules bound to the splitting filter can be transferred to the chemical coupling filter and then reacted with site-specific reagents to chemically modify the bound molecules.

The present invention features a device comprising a splitting filter comprising an addressable array of features, each feature comprising an immobilized capture nucleic acid; and a chemical coupling filter comprising an addressable array of features capable of non-specifically binding nucleic acid-tagged molecules; wherein the splitting filter and the chemical coupling filter are positioned in a confronting relationship so that the features of the splitting filter and the features of the chemical coupling filter are aligned. In some embodiments, at least one of the splitting filter and chemical coupling filter comprises sealing elements bordering each array feature, wherein the sealing elements are positioned between the splitting filter and the chemical coupling filter. In further embodiments, the sealing element is an elastomeric gasket.

In some embodiments, the features on the splitting filter and chemical coupling filter are bordered by sealing elements, wherein the sealing elements of the splitting filter are in mating relationship with the sealing elements of the chemical coupling filter. In further embodiments, the sealing element is an elastomeric gasket.

The present invention also features a method of chemically modifying a plurality of nucleic acid-tagged molecules in a mixture, the method comprising; contacting a first splitting filter with a mixture of nucleic acid tagged molecules, wherein the first splitting filter comprises an addressable array of features, each feature comprising an immobilized capture nucleic acid, and wherein the nucleic acid tagged molecules comprise at least a first hybridization sequence, the contacting providing for splitting the mixture into a plurality of sub-populations of nucleic acid tagged molecules; transferring the sub-populations of nucleic acid tagged molecules to a first chemical coupling filter comprising an array of features capable of non-specifically binding nucleic acid tagged-molecules, the transferring providing for a plurality of immobilized sub-populations of nucleic acid tagged molecules; and reacting the plurality of immobilized sub-population of nucleic acid tagged molecules with a plurality of chemical monomers to chemically modify the plurality of nucleic acid tagged molecules.

In some embodiments, the nucleic acid tags comprise two or more hybridization sequences. In certain embodiments, the method further includes eluting the chemically modified nucleic acid tagged molecules from the first chemical coupling filter and repeating the steps.

The invention also features a kit comprising a splitting filter comprising an addressable array of features, each feature comprising an immobilized capture nucleic acid; and a chemical coupling filter comprising an addressable array of features capable of non-specifically binding nucleic acid-tagged molecules. In some embodiments, the kit further includes a plurality of splitting filters. In some embodiments, the kit further includes a plurality of chemical coupling filters.

In some embodiments, at least one of the splitting filter and chemical coupling filter comprises sealing elements bordering each array feature, wherein the sealing elements are positioned between the splitting filter and the chemical coupling filter. In further embodiments, the sealing element is an elastomeric gasket.

In some embodiments, the features on the splitting filter and chemical coupling filter are bordered by sealing elements, wherein the sealing elements of the splitting filter are in mating relationship with the sealing elements of the chemical coupling filter. In further embodiments, the sealing element is an elastomeric gasket.

These and other objects, advantages, and features of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 shows an exemplary DNA-directed splitting of a library of fragments. The degenerate family of nucleic acid tags in this example is composed of catenated 20 base-pair nucleotide sequences, which are either constant (C₁-C₅) or variable (a₁-j₄). The letters a₁ through j₄ in the variable regions of the DNA fragments denote distinct 20 nucleotide sequences with orthogonal hybridization properties. To carry out the first split, the degenerate family of fragments is passed over a set of ten different affinity resins displaying the sequences a₁^{c}-j₁^{c}, which are complementary to the sequences a₁-j₁ in the first variable region (an exemplary affinity resin is represented by the circle). Ten sub-pools of the original family of fragments result. Each sub-pool of nucleic acid tags is then reacted with a distinct chemical monomer to allow for coupling of the distinct chemical monomer at the chemical reaction site of each nucleic acid tag. The sub-pools are then recombined, and the library is split into a new set of sub-pools based on the sequences a₂-j₂, etc.

FIG. 2 shows an exemplary chemical coupling reaction at the chemical reaction site of a nucleic acid tag. A DEAE-Sepharose resin absorbed nucleic acid tag comprising a chemical reaction site is treated with the NHS ester of FMOC-Alanine in DMP. The FMOC protecting group is removed with piperidine to provide an alanine coupled to the chemical reaction site of the nucleic acid tag. The process can be repeated many times, and with a variety of amino acids at successive steps in order to produce a library of distinct polypeptides.

FIGS. 3A-3D illustrate a method of partition based chemical synthesis using a series of columns to generate a library of distinct chemical compounds.

FIGS. 4A-4F illustrate an exemplary method of carrying out chemical translation on filters according to the present invention.

FIG. 5 is a plan view of a first or separation filter in the device of the invention.

FIGS. 6A-6B show the bottom half of the hybridization chamber (FIG. 6A) and the spin chamber (FIG. 6B), where the pins are used to align filters in the chamber and allow for bolting on of the top half of the chamber.

FIG. 7 shows exemplary amines for peptoid synthesis and observed coupling efficiencies.

### DEFINITIONS

The term “combinatorial library” is defined herein to mean a library of molecules containing a large number, typically between 10³ and 10¹⁵ or more different compounds typically characterized by different sequences of subunits, or a combination of different side chains functional groups and linkages.

The terms “base-specific duplex formation” or “specific hybridization” refer to temperature, ionic strength and/or solvent conditions effective to produce sequence-specific pairing between a single-stranded oligonucleotide and its complementary-sequence nucleic acid strand, for a given length oligonucleotide. Such conditions are preferably stringent enough to prevent or largely prevent hybridization of two nearly-complementary strands that have one or more internal base mismatches. Preferably the region of identity between two sequences forming a base-specific duplex is greater than about 5 bp, more preferably the region of identity is greater than 10 bp.

“Different-sequence small-molecule compounds” refers to small organic molecules, typically, but not necessarily, having a common parent structure, such as a ring structure, and a plurality of different R group substituents or ring-structure modifications, each of which takes a variety of forms, e.g., different R groups. Such compounds are usually non-oligomeric (that is, do not consist of sequences of repeating similar subunits) and may be similar in terms of basic structure and functional groups, but vary in such aspects as chain length, ring size or number, or patterns of substitution.

The term “chemical reaction site” as used herein refers to a chemical component of a nucleic acid tag capable of forming a variety of chemical bonds including, but not limited to; amide, ester, urea, urethane, carbon-carbonyl bonds, carbon-nitrogen bonds, carbon-carbon single bonds, olefin bonds, thioether bonds, and disulfide bonds.

The terms “nucleic acid tag”, “nucleic acid support”, “synthesis-directing nucleic acid tags”, and “DNA-tag” as used herein mean the nucleic acid sequences which each comprise at least (i) a different first hybridization sequence, (ii) a different second hybridization sequence, and (iii) a chemical reaction site. The “hybridization sequences” refer to oligonucleotides comprising between about 3 and up to 50, and typically from about 5 to about 30 nucleic acid subunits. Such “nucleic acid tags” are capable of directing the synthesis of the combinatorial library of the present invention based on the catenated hybridization sequences.

The terms “oligonucleotides” or “oligos” as used herein refer to nucleic acid oligomers containing between about 3 and up to about 50, and typically from about 5 to about 30 nucleic acid subunits. In the context of oligos (e.g., hybridization sequence) which direct the synthesis of the library compounds of the present invention, the oligos may include or be composed of naturally-occurring nucleotide residues, nucleotide analog residues, or other subunits capable of forming sequence-specific base pairing, when assembled in a linear polymer, with the proviso that the polymer is capable of providing a suitable substrate for strand-directed polymerization in the presence of a polymerase and one or more nucleotide triphosphates, e.g., conventional deoxyribonucleotides. A “known-sequence oligo” is an oligo whose nucleic acid sequence is known.

The term “oligonucleotide analog” is defined herein to mean a nucleic acid that has been modified and which is capable of some or all of the chemical or, biological activities of the oligonucleotide from which it was derived. An oligonucleotide analog will generally contain phosphodiester bonds, although in some cases, oligonucleotide analogs are included that may have alternate backbones. (See, E.G., several nucleic acid analogs described in Rawls, C & E News, Jun. 2, 1997, page 35). Modifications of the ribose-phosphate backbone may facilitate the addition of additional moieties such as labels, or may be done to increase the stability and half-life of such molecules. In addition, mixtures of naturally occurring nucleic acids and analogs can be made. Alternatively, mixtures of different nucleic acid analogs, and mixtures of naturally occurring nucleic acids and analogs may be made. The oligonucleotides may be single stranded or double stranded, as specified, or contain portions of both double stranded or single stranded sequence. The oligonucleotide may be DNA, RNA or a hybrid, where the nucleic acid contains any combination of deoxyribo-and ribo-nucleotides, and any combination of bases, including uracil, adenine, thymine, cytosine, guanine, inosine, xathanine hypoxathanine, isocytosine, isoguanine, etc. The term “nucleic acid” as used herein includes oligonucleotide analogs.

The terms “capture nucleic acid”, “capture oligonucleotide”, “and immobilized capture nucleic acid” as used herein refer to a nucleic acid sequence immobilized to a feature of a splitting filter of the device of the invention. In general, the sequence of a capture nucleic acid is complementary to one of the different hybridization sequences (e.g., a₁, b₁, c₁, etc.) of the nucleic acid tags and therefore allows for sequence-specific splitting of a population of nucleic acid tagged molecules into a plurality of sub-populations of distinct nucleic acid tagged molecules.

The term “non-specific binding” as used herein with respect to a “non-specific filter” refer to binding of nucleic acid that does not depend on the nucleic acid sequence applied to the filter. Exemplary materials for non-specific binding include an ion-exchange medium, which is effective to non-specifically capture nucleic acid tagged molecules at one ionic strength, and release the nucleic acid tagged molecules, following molecule reaction, at a higher ionic strength.

The terms “nucleic acid tag-directed synthesis” or “tag-directed synthesis” or “chemical translation” refer to synthesis of a plurality of compounds based on the catenated hybridization sequences of the nucleic acid tags according to the methods of the present invention.

The term “amplifying population of compounds” refers to an increasing population of compounds synthesized according to the catenated hybridization sequences of the nucleic acid tags produced by the iterative methods described herein.

The term “genetic recombination of nucleic acids tags” refers to forming chimeras of nucleic acid tags derived from compounds having one or more desired activities. Chimeras can be formed by genetic recombination, after repeated cycles of enrichment and step-wise synthesis, PCR amplification and step-wise synthesis, partial digestion, reformation and stepwise synthesis to yield a highly enriched subpopulation of nucleic acid tags which are bound to compounds having one or more desired activities.

The term “selection for a desired activity” means evaluating one or more of the plurality of compounds produced by the methods of the invention for the ability to modulate a chemical or biological process of interest, or to bind with high affinity to a macromolecule or target.

The term “ligand” refers to a molecule, antigen, or receptor or enzyme substrate capable of binding specifically and with high affinity to a complementary binding partner.

The terms “tagged compounds”, “DNA-tagged compound”, or “nucleic acid-tagged compound” are used to refer to compounds containing (a) unique nucleic acid tags, each unique nucleic acid tag of each compound includes at least one and preferably two or more catenated different hybridization sequences, wherein the hybridization sequences are capable of binding specifically to complementary immobilized capture nucleic acid sequences, and (b) a chemically reactive reaction moiety that may include a compound precursor, a partially synthesized compound, or completed compound. A nucleic acid tagged compound in which the chemically reactive moiety is a completed-synthesis compound is also referred to as a nucleic acid-tagged compound.

The term “small molecule” refers to a compound having a molecular weight typically between 100 and 800 daltons.

### DETAILED DESCRIPTION OF THE INVENTION

The present invention provides a device and method for synthesizing nucleic acid-templated combinatorial chemical libraries. The device includes a splitting filter having an array of immobilized capture nucleic acids and a chemical coupling filter having an array of non-specific binding features, wherein the plates are positioned to provide for alignment between the capture sites of the first filter and the non-specific binding features of the second filter plate. The molecules bound to the splitting filter can be~transferred to the chemical coupling filter and then reacted with site-specific reagents to chemically modify the bound molecules.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described.

All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a tag” includes a plurality of such tags and reference to “the compound” includes reference to one or more compounds and equivalents thereof known to those skilled in the art, and so forth.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Overview

The present invention provides devices and methods for synthesizing, screening, and amplifying a nucleic acid-templated combinatorial chemical library. The combinatorial chemical library comprise a plurality of species of bifunctional molecules (i.e., nucleic acid tagged molecules) that each comprise a different chemical compound moiety and a unique identifier nucleic acid sequence moiety (i.e., nucleic acid tag), wherein the nucleic acid sequence defines and directs the synthesis of the corresponding chemical compound moiety. Details of the nucleic acid tagged molecules used in the invention and traditional strategies for synthesizing and screening combinatorial nucleic acid tagged compounds are described in PCT patent application WO 00/23458, entitled “DNA Templated Combinatorial Library Chemistry,” published Apr. 27, 2000, and incorporated herein by reference in its entirety.

Described below in greater detail are nucleic-acid tagged molecules used in the method of the invention for producing small-molecule combinatorial libraries, the device of the invention, on which the combinatorial library synthesis is carried out, and methods for using the device of the invention for synthesis of a model combinatorial library.

Nucleic Acid Tagged Molecules and Compounds

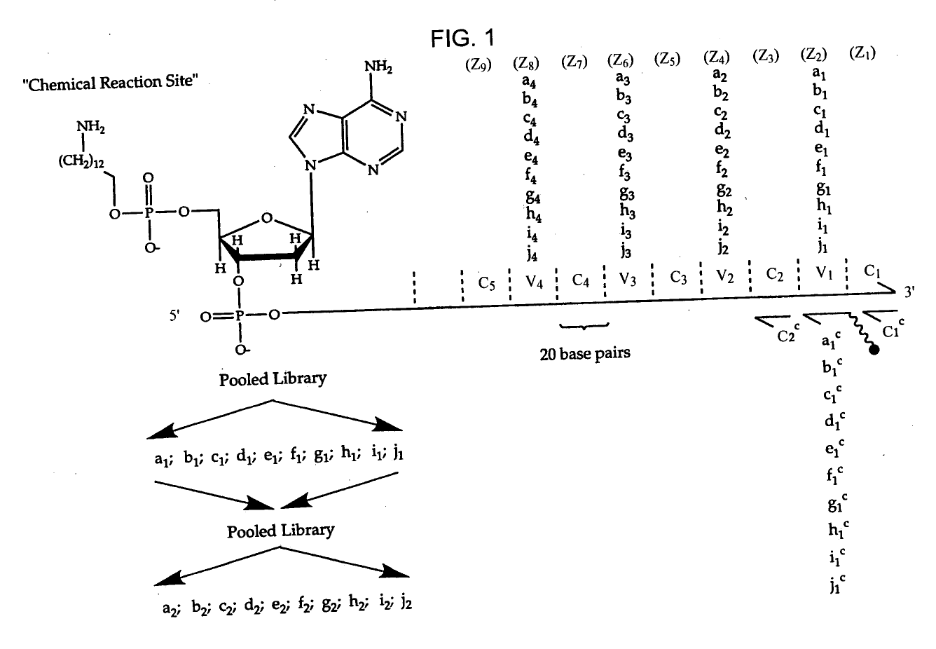

Nucleic acid tagged molecules are ligand tagged compounds having a nucleic acid tag containing at least one, typically two or more different catenated hybridization sequences and an attached, typically a covalently attached, chemical reaction moiety (FIG. 1). The hybridization sequences in any given nucleic acid tag generally differ from the sequences in any other nucleic acid tag. The hybridization sequences of each nucleic acid tag identify the particular chemical monomers that will be used in each successive synthesis step for synthesizing a unique chemical compound attached to the chemical reaction site. As such, hybridization sequences of each nucleic acid tag also identify the order of attachment of the particular chemical monomers to the chemical reaction site.

In general, each hybridization sequence of the nucleic acid tag provides a separate sequence for hybridizing to a complementary capture nucleic acid sequence immobilized to a feature of a splitting filter of the device of the invention. The different hybridization sequences of the nucleic acid tags allow for sequence-specific splitting of a population of nucleic acid tagged molecules into a plurality of sub-populations of distinct nucleic acid tagged molecules. Each sub-population of nucleic acid tagged molecules is then reacted with distinct chemical monomer to allow for coupling of the distinct chemical monomer at the chemical reaction site of each nucleic acid tag.

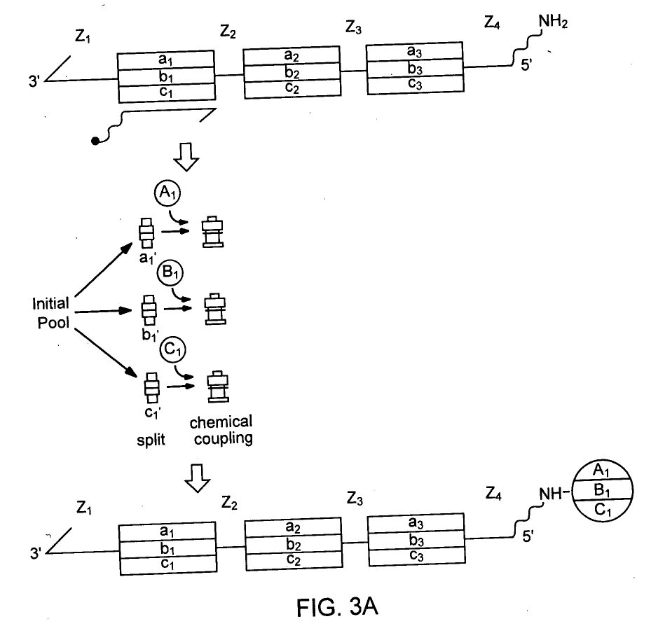

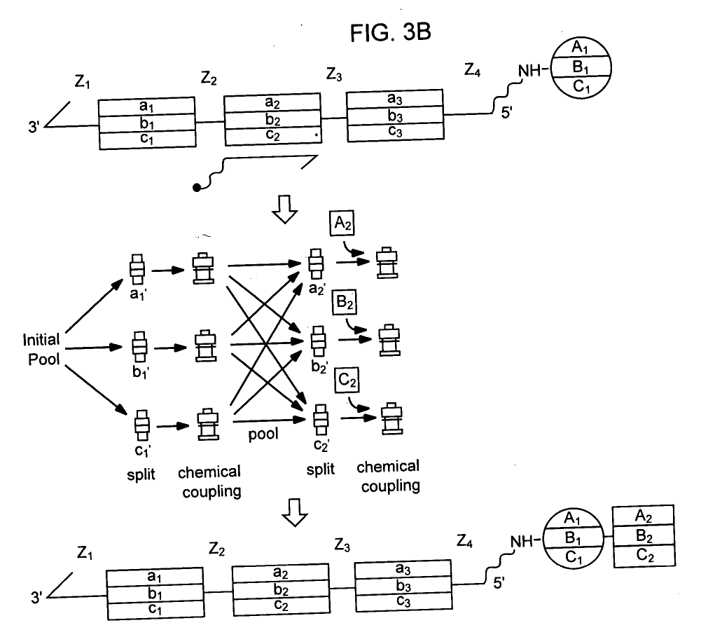

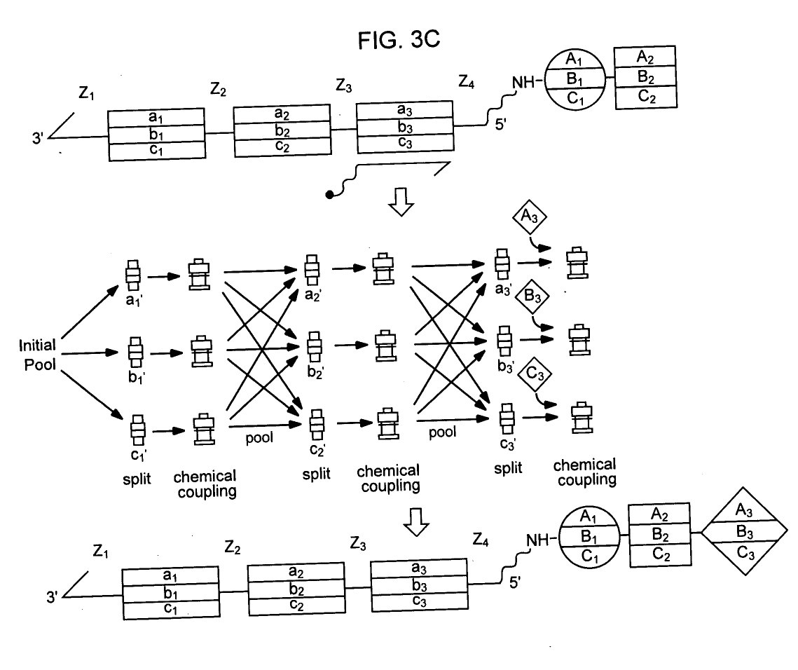

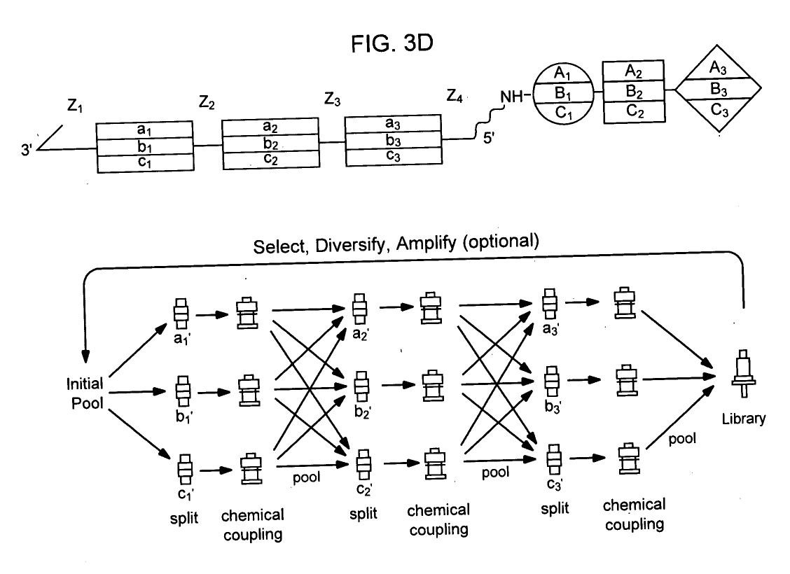

To carry out a first reaction step, the population of nucleic acid tags is “split” into a plurality of sub-populations of distinct nucleic acid tags, e.g., 10 different sub-populations corresponding to the ten different hybridization sequences at the “first” position (V₁, e.g., a₁, b₁, or c₁) in each tag (FIG. 3A, top and middle panels). This is done by contacting the nucleic acid tag-containing molecules with a first group of solid-phase reagents having immobilized capture nucleic acids with sequences complementary to one of the different “first-position” hybridization sequences in the nucleic acid tags (e.g., a₁′, b₁′, or c₁′): These immobilized nucleic acids are sometimes referred to herein as “capture nucleic acid” or “capture oligonucleotides”, and the sequences complementary to a nucleic acid tag sequence referred to as “capture sequences”. This contacting step provides for dividing a population of molecules having different nucleic acid tags into X₁ sub-populations (where X represents the number of different capture sequences used to separate the pooled compounds), where each sub-population of molecules shares at least one common hybridization sequence within the nucleic acid tag.

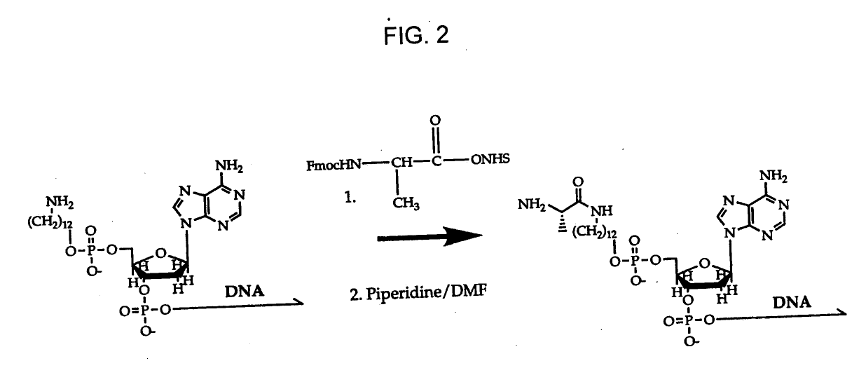

After the first splitting step, the X₁ different nucleic acid tag sub-populations, (e.g., ten different sub-populations of nucleic acid tags as exemplified in FIG. 3A) are reacted with X₁ different chemical monomers (FIG. 3A, middle panel). The reactions are performed such that the identity of each chemical monomer used in the coupling step is directed by the particular “first” position hybridization sequence of the nucleic acid tag in the sub-population. As exemplified in FIG. 3A, the chemical monomer A₁, B₁, or C₁ corresponds to the particular nucleic acid tag hybridization sequence in the “first” position (e.g., a₁, b₁, or c₁). The first chemical coupling step converts the chemical reaction site in each tag to a reagent-specific compound intermediate, by conjugating the particular chemical monomer to the chemical reaction site of each nucleic acid tag sub-population (e.g., A₁, B₁, or C₁, as exemplified in FIG. 2). The result is N₁ different sub-populations of compounds having nucleic acid tags, each sub-population having a different chemical monomer conjugated to the chemical reaction site of each nucleic acid tag sub-population (FIG. 3A, bottom panel). For example, three different populations of nucleic acid tags (as separated by hybridization to a₁, b₁, or c₁ in the “split” step) are represented in the bottom panel of FIG. 3A, where a first sub-population of molecules separated by the a, sequence is modified to contain the chemical monomer A₁, a second sub-population of molecules separated by the b₁ sequence is modified to contain the chemical monomer B₁, and a third sub-population of molecules separated by the a, sequence is modified to contain the chemical monomer C₁. In each instance, a chemical monomer is coupled to the chemical reaction site of the nucleic acid tag-containing compound, where the added chemical monomer provides the reaction site for coupling of an additional monomer in a subsequent step as desired.

Following the first splitting and chemical coupling steps, the X₁ different nucleic acid tag-containing compound sub-populations are contacted with a second group of solid-phase reagents (immobilized capture nucleic acid sequences, e.g., a₂′, b₂′, or c₂′), each having a sequence that is complementary to one of the X₂ different “second-position” hybridization sequences of the nucleic acid tags (e.g., a₂, b₂, or c₂) (FIG. 3B, top and middle panels). As a result, the pooled population of nucleic acid tagged compounds is split into a plurality of X₂ sub-populations of distinct nucleic acid tags. The number of sub-populations in the second step (X₂) may be the same or different than the number of sub-populations resulting from the first stage split (X₁). As above, each sub-population of nucleic acid tagged molecules is determined by the “second-position” hybridization sequence of the nucleic acid tags (e.g., a₂, b₂, or c₂) (FIG. 3B, middle panel).

Each of the different “second-position” sub-populations of nucleic acid tagged compounds is then reacted with one of a second plurality of chemical monomers, a different chemical monomer for each subset (e.g., A₂, B₂, or C₂) (FIG. 3B, middle panel). The result is a X₂ different sub-populations of nucleic acid tags, each population having a different chemical monomer conjugated to the previous chemical monomer of each nucleic acid tag-containing sub-population of molecules (FIG. 3B, bottom panel). For example, as exemplified in the bottom panel of FIG. 3B, nine different sub-populations of nucleic acid tag-containing compounds can be generated, where a first population comprises the chemical monomers A₁ and A₂, a second population comprises the chemical monomers A₁ and B₂, a third population comprises the chemical monomers A₁ and C₂, a fourth population comprises the chemical monomers B₁ and A₂, a fifth population comprises the chemical monomers B₁ and B₂, a sixth population comprises the chemical monomers B₁ and C₂, a seventh population comprises the chemical monomers C₁ and A₂, an eighth population comprises the chemical monomers C₁ and B₂, and a ninth population comprises the chemical monomers C₁ and C₂.

This process of splitting the previously reacted nucleic acid tags into Xₙ different sub-population (where X represents the number of different capture sequences used to separate the pooled compounds and n represents the step number of the synthetic scheme) can be repeated as desired. For example, as illustrated in FIGS. 3C and 3D, the nucleic acid tag-containing compounds can be hybridized with a new set of immobilized capture oligonucleotides, then reacting the Xₙ separated sub-populations of tags with Xₙ different selected chemical monomers. These steps can be repeated until all of the desired reaction steps are performed successively on the reaction sites of the nucleic acid tag-containing compound are complete (FIG. 3C and FIG. 3D). The result is a combinatorial library of X₁×X₂× . . . ×X_{N} different nucleic acid tagged chemical compounds, wherein the particular of hybridization sequences at the N positions (e.g., V₁, V₂, and V₃, see FIG. 1) of the nucleic acid tag of each compound dictates the sequence of chemical monomers of the particular compound.

As exemplified in the top panel of FIG. 3D, twenty-seven different populations of nucleic acid tagged compounds can be generated from the steps as exemplified in FIGS. 3A-3C. The exemplary combinatorial library of compounds includes, for example, a first population comprising the chemical monomers A₁, A₂, and A₃, a second population comprising the chemical monomers A₁, B₂, and A₃, a third population comprising the chemical monomers A₁, C₂, and A₃, a fourth population comprising the chemical monomers B₁, A₂, and A₃, a fifth population comprising the chemical monomers B₁, B₂, and A₃, a sixth population comprising the chemical monomers B₁, C₂, and A₃, a seventh population comprising the chemical monomers C₁, A₂, and A₃, an eighth population comprising the chemical monomers C₁, B₂, and A₃, and a ninth population comprising the chemical monomers C₁, C₂, and A₃, etc.

Nucleic Acid Tag

As exemplified in FIG. 1, the nucleic acid tag is composed of Zₙ (e.g., n=9) regions of different catenated nucleic acid sequences and a chemical reaction site. Five of these regions are denoted C₁ through C₅ and refer to the “constant” or “spacer” sequences that are the same for the nucleic acid tags. The four remaining Z regions are denoted V₁ through V₄ and refer to the “variable” hybridization sequences at the first through fourth positions. In representative embodiments, the V regions and C regions alternate in order from the 3′ end of the nucleic acid tag to the 5′ end of the nucleic acid tag. In certain embodiments, the first Z region is a C region. In other embodiments, the first Z region is a V region. In certain embodiments, the last Z region is a C region. In other embodiments, the last Z region is a V region.

The variable hybridization sequences are generally different for each group of sub-population of nucleic acid tags at each position. In this embodiment, every V region is bordered by two different C regions. As will be appreciated from below, all of the V-region sequences are orthogonal, such that no two V-region sequences cross-hybridize with each other. For example, in an embodiment that comprises nucleic acid tags that include four variable regions and 400 different nucleic acid sequences for each of the four variable regions, there are a total of 1,600 orthogonal nucleic acid hybridization sequences. Such hybridization sequences can be designed according to known methods. For example, where each variable hybridization sequence comprises 20 nucleotides, with a possibility of one of four nucleotides at each position, 20⁴ different sequences are possible. Of the different possible candidates, specific sequences can be elected such that each sequence differs from another sequence by at least 2 to 3, or more, different internal nucleotides.

In general suitable C and V regions comprise from about 10 nucleotides to about 30 nucleotides in length, or more. In certain embodiments, C and V regions comprise from about 11 nucleotides to about 29 nucleotides in length, including from about 12 to about 28, from about 13 to about 27, from about 14 to about 26, from about 14 to about 25, from about 15 to about 24, from about 16 to about 23, from about 17 to about 22, from about 18 to about 21, from about 19 to about 20 nucleotides in length. In representative embodiments C and V regions comprise about 20 nucleotides in length.

A nucleic acid tag can comprise from about 1 to about 100 or more different V regions (hybridization sequences), including about 200, about 300, about 500, or more different V regions. In representative embodiments, a nucleic acid tag comprises from about 1 to about 50 different V regions, including about 2 to about 48, about 3 to about 46, about 4 to about 44, about 5 to about 42, about 6 to about 40, about 7 to about 38, about 8 to about 36, about 9 to about 34, about 10 to about 32, about 11 to about 30, about 12 to about 29, about 13 to about 28, about 13 to about 28, about 14 to about 27, about 15 to about 26, about 16 to about 25, about 17 to about 24, about 18 to about 23, about 19 to about 22, about 20 to about 21 different V regions.

A nucleic acid tag can comprise from about 1 to about 100 or more different C regions (constant sequences), including about 200, about 300, about 500, or more different C regions. In representative embodiments, a nucleic acid tag comprises from about 1 to about 50 different C regions, including about 2 to about 48, about 3 to about 46, about 4 to about 44, about 5 to about 42, about 6 to about 40, about 7 to about 38, about 8 to about 36, about 9 to about 34, about 10 to about 32, about 11 to about 30, about 12 to about 29, about 13 to about 28, about 13 to about 28, about 14 to about 27, about 15 to about 26, about 16 to about 25, about 17 to about 24, about 18 to about 23, about 19 to about 22, about 20 to about 21 different C regions.

The nucleic acid tags are synthesized such that regions Z₁ though Zₙ (e.g., n=9) are linked to each other beginning with Z₁ at the 3′ and continuing in order with the chemical reaction site at the 5′ end following Zₙ. For example, beginning with the 3′ end of the nucleic acid tag, Z₁ is linked to Z₂, Z₂ is linked to Z₃, Z₃ is linked to Z₄, etc., and chemical reaction site is linked to Zₙ at any site on the nucleic acid tag, including the 3′ terminus, the 5′ terminus, or any other position on the nucleic acid tag.

As noted above, a population of nucleic acid tags is degenerate, i.e., almost all of the nucleic acid tags differ from one another in nucleotide sequence. The nucleotide differences between different nucleic acid tags reside entirely in the hybridization sequences (V regions). For example, an initial population of nucleic acid tags can comprise of 400 first sub-populations of nucleic acid tags based on the particular sequence of V₁ of each sub-population. As such, the V₁ region of each sub-population comprises of any one of 400 different 20 base-pair hybridization sequences. Separation of such a population of nucleic acid tags based on V₁ would result in 400 different sub-populations of nucleic acid tags. Likewise, the same initial population of nucleic acid tags can also comprise of 400 second subpopulations of nucleic acid tags based on the particular sequence of V₂ of each subpopulation, wherein the second sub-populations are different than the first subpopulations.

In the exemplary population of nucleic acid tags demonstrated in FIG. 1, the first few of the first hybridization sequences are denoted as a₁, b₁, c₁ . . . j₁, in the V₁ region of the different nucleic acid tags. Likewise, the first few of the second hybridization sequences are denoted as a₂, b₂, c₂ . . . j₂, in the V₂ region of the different nucleic acid tags. The first few of the third hybridization sequences are denoted as a₃, b₃, c₃ . . . j₃, in the V₃, etc.

In certain embodiments, the nucleic acid tags share the same twenty base-pair sequence for designated spacer regions while having a different twenty base-pair sequence between different spacer regions. For example, the nucleic acid tags comprise the same C₁ spacer region, the same C₂ spacer region, and the same C₃ spacer region, wherein C₁, C₂, and C₃ are different from one another.

Thus each 180 nucleotide long nucleic acid tag consists of an ordered assembly of 9 different twenty base-pair regions comprising the 4 variable regions (a₁, b₁, c₁ . . . d₅, e₅, f₅, . . . h₁₀, i₁₀, j₁₀) and the 5 spacer regions (z₁ . . . z₁₁) in alternating order. The twenty base-pair regions have the following properties: (i) micromolar concentrations of all the region sequences hybridize to their complementary DNA sequences efficiently in solution at a specified temperature designated Tm, and (ii) the region sequences are orthogonal to each other with respect to hybridization, meaning that none of the region sequences cross-hybridizes efficiently with another of the region sequences, or with the complement to any of the other region sequences, at the temperature Tm.

The degenerate nucleic acid tags can be assembled from their constituent building blocks by the primerless PCR assembly method described by Stemmer et al., Gene 164(1):49-53 (1995).

Chemical Reaction Site

As noted above the nucleic acid tags further comprise a chemical reaction site any site, including the 3′ terminus, the 5′ terminus, or any other position on the nucleic acid tag. In some embodiments, the chemical reaction site can be added by modifying the 5′ alcohol of the 5′ base of the nucleic acid tag with a commercially available reagent which introduces a phosphate group tethered to a linear spacer, e.g., a 12-carbon chain terminated with a primary amine group (e.g., as available from Glen Research, or numerous other reagents which are available for introducing thiols or other chemical reaction sites into synthetic DNA).

The chemical reaction site is the site at which the particular compound is synthesized dictated by the order of V region sequences of the nucleic acid tag. An exemplary chemical reaction site is a primary amine. Many different types of chemical reaction sites in addition to primary amines can be introduced at any site, including the 3′ terminus, the 5′ terminus, or any other position on the nucleic acid tag. Exemplary chemical reaction sites include, but are not limited to, chemical components capable of forming amide, ester, urea, urethane, carbon-carbonyl bonds, carbon-nitrogen bonds, carbon-carbon single bonds, olefin bonds, thioether bonds, and disulfide bonds. In the case of enzymatic synthesis, co-factors may be supplied as are required for effective catalysis. Such co-factors are known to those of skill in the art. An exemplary cofactor is the phosphopantetheinyl group useful for polyketide synthesis.

Filter-Array Device

The device of the invention includes a first splitting filter having an array of capture sites, which capture sites are provided as defined addressable features of the array. The capture sites are composed of immobilized capture nucleic acids, where capture nucleic acids at different features of the array contain a sequence that hybridizes to (e.g., is complementary to) a different nucleic acid tag subsequence (e.g., a different hybridization sequence in the V₁ region of the nucleic acid tag). The device also includes a first chemical coupling filter having an array of features composed of non-specific binding sites, which features are in alignment with the features containing the capture sites of the first filter so as to provide for transfer of bound nucleic acid tags from the first filter to the second filter (e.g., when an eluting fluid is passed through the first splitting filter and to the first chemical coupling filter).

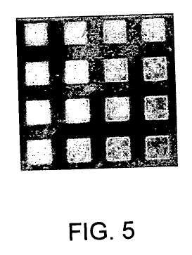

In certain embodiments, each filter, as seen in FIG. 5 includes a plurality of features or fluid retaining structures (light circles in the figure), each of which can be bordered by a sealing element, such as an elastomeric gasketing material (dark areas in the figure), that serves to confine liquid in each feature and to provide a seal for each feature when the splitting filter and chemical coupling filter are placed in an aligned, confronting relationship during transfer of nucleic acid tagged molecules from the splitting filter to the chemical coupling filter, such that the features of the splitting filter are in fluid communication with a corresponding features of the chemical coupling filter. In such embodiments, sealing element of each filter (e.g., splitting filter or chemical coupling filter) is in mating relationship with the sealing element of the other filter.

In other embodiments, at least one of the splitting filter and the chemical coupling filter comprises a sealing elements, such as an elastomeric gasketing material, bordering each feature that serves to confine liquid in each feature and to provide a seal for each feature when the first and second plates are placed in an aligned, confronting position during transfer of nucleic acid tagged molecules from the first splitting filter to the first chemical coupling filter.

In general each filter generally comprises a plurality of spatially addressable features (e.g., more than about 10, more than about 50, more than about 100, more than 200, features, usually up to about 500 features, about 1,000 features, about 10,000 features, about 20,000 features, about 100,000 features or more, including about 24 features, about 48 features, about 96 features, about 192 features, about 384 features and about 1536 features). The subject arrays may be an array of features, each feature corresponding to a “fluid-retaining structure”, e.g., a well, wall, liquid impermeable barrier, or the like. Such arrays are well known in the art, and include 24-well, 48-well, 96-well, 192-well, 384-well and 1536-well microtiter plates, or multiple thereof. In certain embodiments, the features are delineated by a liquid impermeable chemical boundary, and, accordingly, the array substrate may be planar and contain features containing a liquid impermeable boundary. Other fluid retaining structures are well known in the art and include physical and chemical barriers. In one embodiment, the fluid retaining structure is formed by a bead of liquid impermeable material, e.g., a bead of a viscose silicone material, around a fluid-retaining area.

For example, each feature of the splitting filter comprises a different immobilized capture oligonucleotide specific for a unique hybridization sequence of a nucleic acid tag. As such, the immobilized oligonucleotides of each feature comprise a nucleic acid sequence complementary to one of the different “first-position” hybridization sequences in the nucleic acid tags (e.g., a₁′, b₁′, or c₁′). When a population of nucleic acid tags is contacted with the first filter plate, the population of nucleic acid tags is “split” into a plurality of sub-populations of distinct nucleic acid tags coming into contact with the corresponding complementary hybridization sequence immobilized on the splitting filter. The splitting of the initial population of nucleic acid tags using the first splitting filter corresponds to the first split of the method using a plurality of columns as described above (FIG. 3A).

The chemical coupling filter comprises an array of features that is a replica of the array in the splitting filter, so that when the chemical coupling filter is positioned parallel to the splitting filter (e.g., the splitting filter is placed above the chemical coupling filter) the features of the splitting filter are in corresponding alignment (e.g., face to face) with the features of the chemical coupling filter. As such, each feature in the splitting filter confronts and is in registry with a corresponding feature in the chemical coupling filter. Furthermore, the sealing element (e.g., a gasket) that borders each feature in the filter forms a sealed chamber between each pair of confronting feature, so that fluid passed through one filter is confined to these chambers, insuring that molecules released from a feature in the splitting filter are confined to contact with the confronting feature in the second chemical coupling filter.

The chemical coupling filter comprises a suitable medium or coating for non-specifically capturing molecules passed through the filter and which are suitable for chemical reactions performed on the filter. By “non-specifically capturing” is meant non-sequence specific binding of the molecules. Exemplary materials for the second filter include an ion-exchange medium, which is effective to non-specifically capture nucleic acid tagged molecules at one ionic strength, and release the nucleic acid tagged molecules, following molecule reaction, at a higher ionic strength. One suitable ion-exchange medium suitable for this application is formed as described in the example below. The features of the chemical coupling filter are then contacted with different chemical monomers to conjugate the different chemical monomers with the different sub-populations of nucleic acid tags. The chemical coupling filter is also referred to herein as a “non-specific filter”. The chemical coupling of nucleic acid tags using the first chemical coupling filter corresponds to the first coupling step of the method using a plurality of columns as described above (FIG. 3A).

For use in DNA-template synthesis, which may involve more than one reaction, e.g., a 3-6 synthesis steps, the device includes a separate splitting filter and a separate corresponding chemical coupling filter for each reaction step. Therefore, the array of immobilized complementary oligonucleotides of each successive splitting filter correspond to one of the Vₙ variable sequences of a nucleic acid tag, where n represents nth synthetic step in the reaction. Thus, for example, where the nucleic acid tagged molecules each include three different variable hybridization sequences (e.g., a₁, b₁, c₁, d,₁, . . . , a₂, b₂, c₂, d₂, . . . , a₃, b₃, c₃, d₃) each containing one of 400 different sequences, the device would include 3 different splitting filter plates, each containing 400 features, and each feature comprising an immobilized oligonucleotide probe complementary to one of the 400 different variable sequences of the nucleic acid tags, corresponding to a given reaction step.

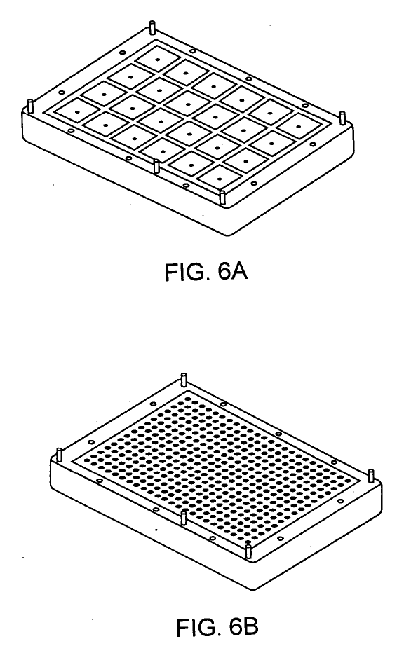

FIG. 6A and FIG. 6B show an exemplary transfer apparatus for use in transferring nucleic acid tagged molecules from a first filter to second filter. FIG. 6B shows a 384 well Delrin plate forming the bottom of a transfer chamber. Transfer medium and transfer conditions are similar to those described in the PCT application WO 00/23458, incorporated herein by reference in its entirety.

Synthesis Methods

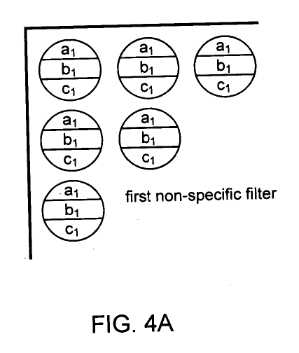

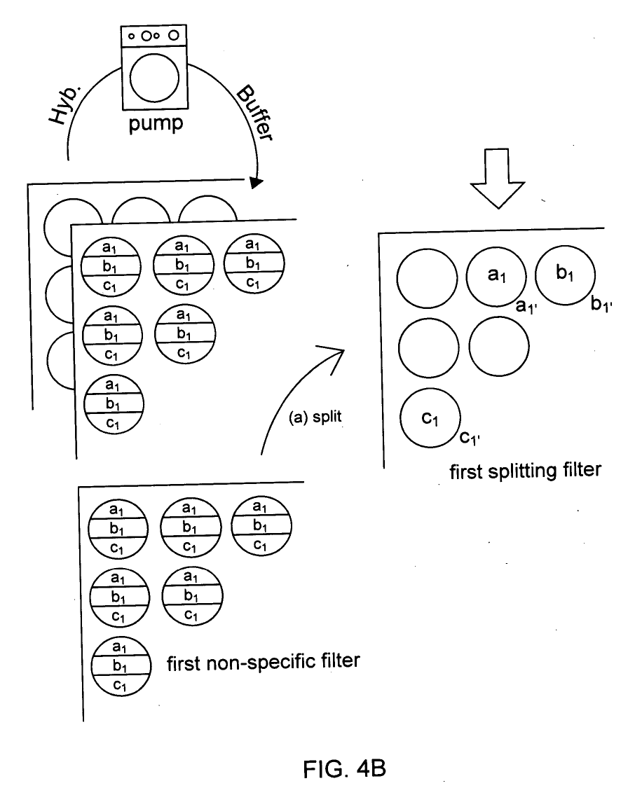

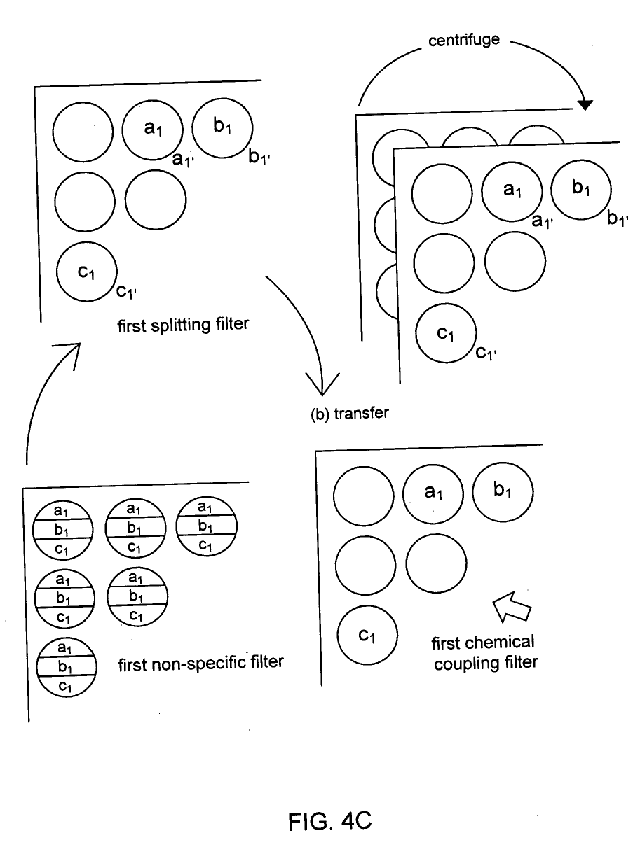

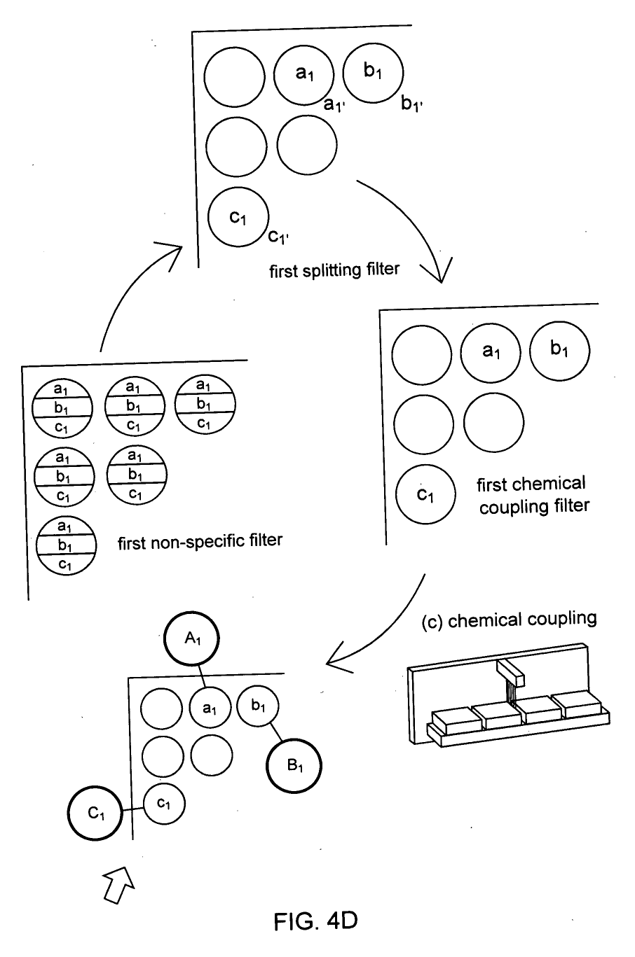

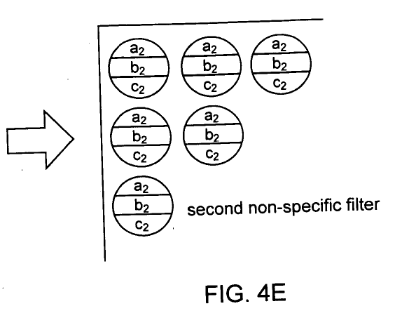

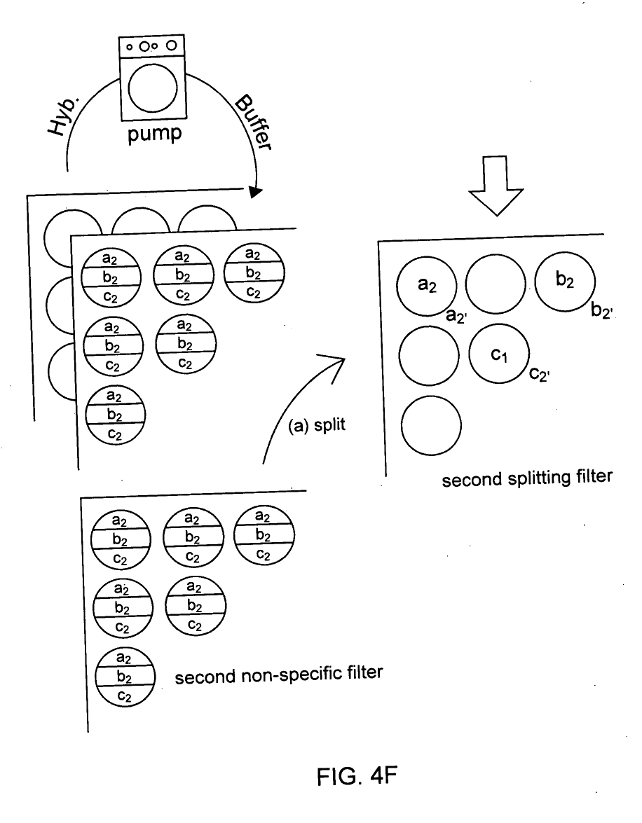

In general, nucleic acid tagged molecules are applied to the splitting filter so that all of the nucleic acid tagged molecules in the population are accessible to all of the features on the first splitting filter, and allowed to hybridize with the immobilized complementary capture nucleic acids of the features under conditions that favor hybridization (FIG. 4A and FIG. 4B). At this point, each sub-population of nucleic acid tagged molecules having a particular sequence at the first variable position will hybridize to the complementary immobilized oligonucleotide of a particular feature.

The first splitting filter is then placed in transfer alignment with the first chemical coupling filter, as above, and a suitable eluting liquid is passed through the two filters, releasing molecules from the first-filter features and capturing the released molecules on the corresponding features of the first chemical coupling filter (FIG. 4C). The first chemical coupling filter is then removed, and a different chemical monomer (building-block) is added to each feature of the first chemical coupling filter under suitable reaction conditions for carrying out the desired chemical modification of each of the sub-population of nucleic acid tags (FIG. 4D). Following the chemical coupling of the chemical monomers to the chemical reaction sites of the nucleic acid tags, the nucleic acid tagged molecules are released from the first chemical coupling filter (FIG. 4E), and applied to the second splitting filter plate, for splitting of the nucleic acid tagged molecules into different sub-populations of nucleic acid tagged molecules (FIG. 4F). The nucleic acid tagged molecules are applied to the second splitting filter so that all of the nucleic acid tagged molecules in the population are accessible to all of the features on the second splitting filter, and allowed to hybridize with the immobilized complementary capture nucleic acids of the features under conditions that favor hybridization. In some embodiments, the released nucleic acid tagged molecules are pooled prior to contacting the second splitting filter. The process is repeated until each of the N synthetic steps have been carried out, producing a library equal in size to X₁×X₂× . . . ×X_{N}, where X is the total number of reactions performed on each filter plate, e.g., 384, and N is the number of different reaction steps, e.g., 3-6.

General methods for the synthesis of small molecule libraries using DNA-template synthesis is described in WO 00/23458, incorporated herein by reference in its entirety. The method is further detailed in FIG. 3A to FIG. 3D. Further details regarding the selection and chemical manipulation described herein may be found in one or more of the reference cited above, all incorporated herein by reference.

Additional procedures for carrying out template-DNA synthesis using the device of the invention are described below.

Use of Device for Synthesis of Capped a Tri-Peptoid Library

As an exemplary synthetic method, the use of the device of the invention for DNA-templated synthesis will be described for the synthesis of a capped tri-peptoid library, involving four chemical coupling steps (reaction steps) and 384 different chemical modifications at each chemical coupling step. Although the peptoid backbone resembles a peptide backbone, the side-chain substituents are attached through the amide nitrogen, rather than through the alpha carbon. This arrangement endows peptoids with several advantages relative to peptides. First, the amide nitrogen is removed as a hydrogen-bond donor. Second, peptoids can be synthesized using primary amines as the diversity element, rather than using protected amino acids. Commercially available primary amines are extremely abundant, unlike amino acids. Third, peptoids are not recognized by proteases, and are therefore extremely resistant to metabolic breakdown in animals. Finally, the synthesis of peptoids on a gram scale is both inexpensive and easy. A number of combinatorial peptoid libraries have been described in the literature.

In one exemplary embodiment, synthesis of a peptoid library employs 384 primary amines and 384 capping reagents. Exemplary amines that are commercially available include 400 suitable primary amines less than 135 daltons, 213 isocyanates less than 199 daltons, 400 carboxylic acids less than 144 daltons, 200 aldehydes less than 148 daltons, and 200 ketones less than 129 daltons. From among these, 384 amines can be selected, along with a mixed assortment of 384 capping reagents.

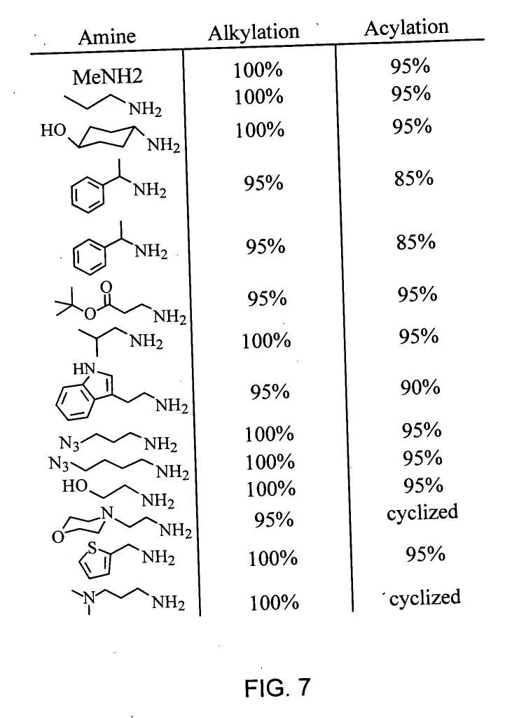

The 384 selected amines are aliquoted into sixteen 24 well plates as 2 M DMSO solutions, and stored at −70° C. Similarly, the capping reagents are stored in 24-well plates as 2M DMF solutions. In order to format reagents for combinatorial library synthesis, the chemical monomer (building-block) stock solutions in sixteen 24-well plates are reformatted into four 96-well plates, which are then used to generate a single 384-well plate. The reformatting transfers are carried out with, for example, a Bio-Tek robot. Exemplary amines, along with observed coupling efficiencies in alkylation and acylation are shown in FIG. 7.

Four different 384-feature splitting filters are required for nucleic acid tag splitting and combinatorial library synthesis. The sequences on these array filters are from a set of 10,000 twenty-mer oligonucleotides that do not cross hybridize. 1536 twenty-base oligonucleotides with 5′ amine modifications are synthesized in 96-well plates, and purified over 96-well Sep-Pak cartridges (Waters, Milford Mass.). The capture nucleic acids are then immobilized in addressable manner on the four splitting filters.

For splitting the population of nucleic acid tagged molecules into distinct sub-populations, the nucleic acid tagged molecules are applied in mixture directly to the splitting filter so that all of the nucleic acid tagged molecules in the population are accessible to all of the features on the first splitting filter, and allowed to hybridize with the immobilized complementary capture nucleic acids of the features under conditions that favor hybridization (FIG. 4A). Depending on the sequence of the complementary capture nucleic acid immobilized at a distinct feature, a particular sub-population of nucleic acid tags will hybridize to the feature and the remaining non-complementary nucleic acid tags will be washed away. As a result, the nucleic acid tags are split into different sub-populations of nucleic acid tags, where each sub-population is present at a different feature of the splitting filter(FIG. 4B). The distinct sub-populations of nucleic acid tags (e.g., a₁, b₁, and c₁) and corresponding capture nucleic acids (e.g., a₁′, b₁′, and c₁′) at each feature of the splitting filter in FIG. 4B are represented by the different labels

Following splitting of the initial population of nucleic acid tags into distinct sub-population of nucleic acid tags based on the hybridization sequences in the first position of the nucleic acid tags, the distinct sub-populations are transferred to a first coupling filter for chemical coupling of distinct chemical monomers to the chemical reaction sites of the nucleic acid tags (FIG. 4C). The first splitting filter is paired with an anion-exchange first chemical coupling filter and the two filters are compressed between two 384-well plates, one of which contains 150 μl of 50% DMF. The plates are then centrifuged so that the DMF in the 384 independent solvent channels passes through the paired filters, denaturing the hybridized nucleic acid duplexes and promoting absorption of the nucleic acid tags released from the first splitting filter and onto the first anion-exchange chemical coupling filter. In certain embodiments the non-specific binding filter and the chemical coupling filter comprise the same non-specific material, e.g., anion-exchange material. Therefore, in such embodiments, a filter that is suitable for use as a non-specific filter is also suitable for use as a chemical coupling filter.

The transferred arrays of distinct sub-populations of nucleic acid tags are then chemically reacted with distinct chemical monomers (peptoid building block) (e.g., A₁, B₁, and C₁) (FIG. 4D). The identity of the particular chemical monomer reacted with each sub-population of nucleic acid is dictated by the particular hybridization sequence in the first position of the nucleic acid tag. The different chemical monomers conjugated to the chemical reaction sites of the sub-populations of nucleic acid tags are represented in FIG. 4D (e.g., A₁, B₁, and C₁).

Following the first chemical conjugation, the nucleic acid tagged molecules are eluted form the first chemical conjugation filter and brought into contact with a second splitting filter. The nucleic acid tagged molecules are applied to the second splitting filter so that all of the nucleic acid tagged molecules in the population are accessible to all of the features on the second splitting filter, and allowed to hybridize with the immobilized complementary capture nucleic acids of the features under conditions that favor hybridization. This is accomplished by pairing the first chemical coupling filter with the second splitting filter and flowing high iconic strength buffer cyclically over all 384 features in the hybridization chamber (FIG. 6). In some embodiments, the released nucleic acid tagged molecules are pooled prior to contacting the second splitting filter.

Therefore, the different sub-populations of nucleic acid tags bound to each feature of the first non-specific filter are brought into contact with the corresponding feature of the second splitting filter. Depending on the sequence of the complementary capture nucleic acids immobilized at each distinct feature, a particular sub-population of nucleic acid tags will hybridize to the immobilized capture nucleic acid directed by the hybridization sequence in the second position of the nucleic acid tags. As a result, the nucleic acid tags are split again into different sub-populations of nucleic acid tags, where each sub-population is present at a different feature of the second slitting filter. The distinct sub-populations resulting from the second split will generally be different than the sub-populations resulting from the first split. The distinct sub-populations of nucleic acid tags (e.g., a₂, b₂, and c₂) and corresponding capture nucleic acids (e.g., a₂′, b₂′, and c₂′) at each feature of the splitting filter in FIG. 4E are represented by the different labels.

The chemical coupling and splitting cycles are repeated, as shown, until all of the reaction steps, e.g., the four reaction steps in the capped tri-peptoid example, have been carried out to generate a combinatorial library of compounds.

Kits

Also provided by the subject invention are kits for practicing the subject methods, as described above. The subject kits include at least a first splitting filter having an array of immobilized capture nucleic acids each specific for a unique hybridization sequence of a nucleic acid tag and a first chemical coupling filter having an array of non-specific binding sites in alignment with the features of the first splitting filter allowing for transfer of bound nucleic acid tags from the first splitting filter to the first chemical coupling filter when an eluting fluid is passed through the two filters. The individual binding sites in the chemical coupling filter can then be reacted with chemical monomers to chemically modify each of the molecules bound to such sites in a specific manner. In some embodiments, the kit further comprises a plurality of splitting filters. In some embodiments, the kit further comprises a plurality of chemical coupling filters.

In some embodiments, the kits contain programming means to allow a robotic system to perform the subject methods, e.g., programming for instructing a robotic pipettor to add, mix and remove reagents, as described above. The various components of the kit may be present in separate containers or certain compatible components may be precombined into a single container, as desired.

The subject kits may also include one or more other reagents for preparing or processing a nucleic acid tag according to the subject methods. The reagents may include one or more matrices, solvents, sample preparation reagents, buffers, desalting reagents, enzymatic reagents, denaturing reagents, where calibration standards such as positive and negative controls may be provided as well. As such, the kits may include one or more containers such as vials or bottles, with each container containing a separate component for carrying out a sample processing or preparing step and/or for carrying out one or more steps of a combinatorial library synthesis protocol suing nucleic acid tags.

In addition to above-mentioned components, the subject kits typically further include instructions for using the components of the kit to practice the subject methods, i.e., to synthesize a combinatorial library using the subject device and/or screening a combinatorial library according to the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject database, programming and instructions, the kits may also include one or more control analyte mixtures, e.g., two or more control samples for use in testing the kit.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Preparation of Gasketed Patterns on Filters

The gaskets are made by photocuring a form-in-place elastomeric gasketing material, which is initially a resin. The resin is cured by illumination with ultraviolet light passing through a mask in contact with the membrane. Resin behind the dark areas of the mask (the 384 wells) remains a liquid, and is removed with acetone.

A mask of an array of 3 mm diameter holes at 4.5 mm spacing is drawn using Adobe Illustrator and printed on a transparency using a black-and-white laser printer. The mask is placed on top of the filter, and both are then placed under an Oriel 500 W mercury arc lamp UV source. The filter is then exposed for 15 sec at 500 W. The mask is removed from the filter, and the unpolymerized excess is washed out of the wells using acetone.

### Example 2

### Preparation of Chemical Conjugation Filter

A 4.25 cm diameter Whatman GF/D glass-fiber filter is soaked in 1.5 ml of Norland Optical Adhesive 74 or 81 or EMI EMCAST 1852 clear and cured under a 500W UV light source for 15-30 seconds, to produce the gasket pattern on the filter. The unpolymerized material is removed by pulling acetone through the filter with a vacuum. The filter is then incubated with 1 ml of trichlorosilane at 60° C. for 1 hour. The filter is washed with methanol and incubated in 1 ml of a quaternary amine methacrylate:bisacrylamide solution (33 mg methylene bisacrylamide, 500 μl (3-acrylamidopropyl)trimethylammonium chloride, 15 mg AIBN, 15 μl TEMED, 515 μl methanol) for 12-18 hours at 60° C. The filter is then washed with 1:1 methanol:chloroform.

Alternatively, a 4.25 cm diameter Pall Biodyne B membrane (manufactured by 7 Pall) is soaked in 1 ml of Norland Optical Adhesive 74 or 81 or EMI EMCAST 1852 clear and cured under a 500W UV light source for 15-30 sec, the produce the gasket pattern on the filter. The Biodyne B is a nylon membrane derivatized with a quaternary amine.

Another method of preparing a chemical conjugation filter includes incubating a Millipore 231 cellulose filter in 5 ml of a solution of 1M (3-bromopropyl)trimethylammonium bromide and 2M 3-bromopropionic acid in 0.1M sodium hydroxide for twelve hours at room temperature. The filter is subsequently washed with water and immersed in 4 ml of EMCAST 1852, a commercially available, UV-curable polymer. The filter is covered with a mask patterned with an array of 384 squares and is exposed to a UV light source for 5 to 15 seconds. The unpolymerized excess is removed by washing with acetone.

### Example 3

### Preparation of Splitting Filter

EMCAST 1852, a UV-curable polymer, is embedded in a Millipore 231 cellulose filter, and the masked filter is exposed to a UV light source for 5 to 15 seconds. The unpolymerized excess is removed by washing with acetone.

A linker is prepared from polyethylene glycol (1000) bisepoxide. 5 g of the bisepoxide are mixed with 3.3 g of sodium azide in a solution of 8 ml water and 4.6 ml acetic acid for 30 minutes at room temperature. 20 ml of 10% sodium hydroxide are added, and the reaction is extracted into 20 ml of methylene chloride three times. The organic fractions are dried over sodium sulfate and concentrated in vacuo to produce a white oil in 75% yield. The bisazide PEG is resuspended in 50 ml of methylene chloride and 37.5 ml of 0.1M phosphoric acid. 0.9 g of triphenyl phosphine is added under nitrogen, and the reaction is incubated for 14 hours. 50 ml of a 10% sodium hydroxide solution is added to the reaction, and the reaction is extracted three times with 50 ml of methylene chloride. The organic fractions are dried and concentrated to produce an azide-amine linker.

30 ul of a 10% (w:v) carbonyl diimidazole solution is added is deposited in each well for 1 hour at room temperature. The filter is washed with acetone, and 30 ul of a 50 uM solution of the azide-amine PEG linker is deposited in each well of the filter. The filters are placed at room temperature overnight. The filters are washed with acetone, water, and DMSO. 20 ul of 5 uM alkyne-terminated 20-mer oligonucleotide in a 1:1 DMSO:water mixture with 600 uM sodium ascorbate and 810 uM Cu(I)-TBTA (trisbenzyltriazolylamine) are deposited in each well, and the solution is incubated at room temperature for 30 minutes. The wells are then evacuated using a vacuum manifold and washed with DMSO and water.

Alternatively, a linker is prepared from polyethylene glycol (1000) bisepoxide. 5 g of the bisepoxide are mixed with 3.3 g of sodium azide in a solution of 8 ml water and 4.6 ml acetic acid for 30 minutes at room temperature. 20 ml of 10% sodium hydroxide are added, and the reaction is extracted into 20 ml of methylene chloride three times. The organic fractions are dried over sodium sulfate and concentrated in vacuo to yield a white oil in 75% yield.

The bisazide PEG is resuspended in a solution of 50 ml of methylene chloride and 50 ml of saturated sodium bicarbonate. This solution is stirred on ice for 10 minutes. 1 g of triphosgene in 10 ml of methylene chloride is added and stirred for 10 minutes. The organic fraction is separated, and the aqueous layer is extracted three times with methylene chloride. The organic fractions are pooled, dried, and concentrated to produce an azide-isocyanate linker.

30 ul of a solution of 50 uM azide-isocyanate linker and 50 uM dibutyl tin diacetate in DMF is deposited in each well of the filter, and the filters are placed at 50C overnight. The filters are washed with DMF. 20 ul of 5 uM alkyne-terminated 20-mer oligonucleotide in a 1:1 DMSO:water mixture with 600 uM sodium ascorbate and 810 uM Cu(I)-TBTA (trisbenzyltriazolylamine) are deposited in each well, and the solution is incubated at room temperature for 30 minutes. The wells are then evacuated using a vacuum manifold and washed with DMSO and water.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A device comprising;
a splitting filter comprising an addressable array of features, each feature comprising an immobilized capture nucleic acid; and
a chemical coupling filter comprising an addressable array of features capable of non-specifically binding nucleic acid-tagged molecules;
wherein the splitting filter and the chemical coupling filter are positioned in a confronting relationship so that the features of the splitting filter and the features of the chemical coupling filter are aligned.

2. The device of claim 1, wherein at least one of the splitting filter and chemical coupling filter comprises sealing elements bordering each array feature, wherein the sealing elements are positioned between the splitting filter and the chemical coupling filter.

3. The device of claim 2, wherein the sealing element is an elastomeric gasket.

4. The device of claim 1, wherein the features on the splitting filter and chemical coupling filter are bordered by sealing elements, wherein the sealing elements of the splitting filter are in mating relationship with the sealing elements of the chemical coupling filter.

5. The device of claim 4, wherein the sealing element is an elastomeric gasket.

6. A method of chemically modifying a plurality of nucleic acid-tagged molecules in a mixture, said method comprising;
(a) contacting a first splitting filter with a mixture of nucleic acid tagged molecules, wherein the first splitting filter comprises an addressable array of features, each feature comprising an immobilized capture nucleic acid, and wherein the nucleic acid tagged molecules comprise at least a first hybridization sequence, the contacting providing for splitting the mixture into a plurality of sub-populations of nucleic acid tagged molecules;
(b) transferring the sub-populations of nucleic acid tagged molecules to a first chemical coupling filter comprising an array of features capable of non-specifically binding nucleic acid tagged-molecules, the transferring providing for a plurality of immobilized sub-populations of nucleic acid tagged molecules; and
(c) reacting the plurality of immobilized sub-population of nucleic acid tagged molecules with a plurality of chemical monomers to chemically modify the plurality of nucleic acid tagged molecules.

7. The method of claim 6, wherein the nucleic acid tags comprise two or more hybridization sequences.

8. The method of claim 7, further comprising eluting the chemically modified nucleic acid tagged molecules from the first chemical coupling filter and repeating steps (a)-(c).

9. A kit comprising:
a splitting filter comprising an addressable array of features, each feature comprising an immobilized capture nucleic acid; and
a chemical coupling filter comprising an addressable array of features capable of non-specifically binding nucleic acid-tagged molecules.

10. The kit of claim 9, wherein at least one of the splitting filter and chemical coupling filter comprises sealing elements bordering each array feature, wherein the sealing elements are positioned between the splitting filter and the chemical coupling filter.

11. The kit of claim 10, wherein the sealing element is an elastomeric gasket.

12. The kit of claim 9, wherein the features on the splitting filter and chemical coupling filter are bordered by a sealing elements, wherein the sealing elements of the splitting filter are in mating relationship with the sealing elements of the chemical coupling filter.

13. The kit of claim 12, wherein the sealing element is an elastomeric gasket.

14. The kit of claim 9, further comprising a plurality of splitting filters.

15. The kit of claim 9, further comprising a plurality of chemical coupling filters.
